(19) United States
(12) Patent Application Publication
(10) Pub. No.: US 2009/0118749 A1
(43) Pub. Date: May 7, 2009

(54) Pyloric Devices and Methods

(75) Inventors: Tidhar Shalon, Tel-Aviv (IL); Tadmor Shalon, Palo Alto, CA (US); Guy Kotlizky, Kfar-Shemaryahu (IL)

Correspondence Address: MARTIN D. MOYNIHAN d/b/a PRTSI, INC., P.O. BOX 16446, ARLINGTON, VA, 22215, US

(73) Assignee: SVIP 2 LLC, Palo Alto, CA (US)

(21) Appl. No.: 12/224,873

(22) PCT Filed: Mar. 19, 2007
(86) PCT No.: PCT/IL2007/000356

§ 371 Date: Sep. 9, 2008

Related U.S. Application Data
(60) Provisional application No. 60/783,406, filed on Mar. 20, 2006.

(51) Int. Cl. A61B 17/122

(52) U.S. Cl. 606/157

(57) ABSTRACT

A gastrointestinal device is provided. The device includes a band sized and configured for residing in or around a pyloric sphincter region of the subject. The band is functional in maintaining the pyloric sphincter at a fixed opening size.

### FIELD AND BACKGROUND OF THE INVENTION

The present invention relates to devices and methods which can be used to alter the pyloric opening. More particularly, the present invention relates to devices for fixing an opening size of the pyloric sphincter and to methods of using such devices to alter satiety, treat a variety of gastrointestinal disorders such as obesity, gastroparesis, gastroesophageal reflux disease (GERD), or precondition a subject for bariatric surgery.

During the past 20 years, obesity among adults has risen significantly in the United States. The latest data from the National Center for Health Statistics show that 30 percent of U.S. adults 20 years of age and older—over 60 million people—are obese. Obesity requires long-term management; the goal of treatment is weight loss to improve, prevent occurrence of, or eliminate related health problems.

Numerous approaches for the treatment of obesity are known in the art, including drug treatment, surgical procedures and implantable devices.

Drugs for treatment of obesity fall into three general categories, appetite altering drugs such as dexfenfluramine or sibutramine which suppresses appetite by altering neurotransmitter release or uptake in the brain; metabolism-changing drugs such as Orlistat which prevents the action of lipases (enzymes that break down fat) produced in the pancreas; and drugs that increase energy output (‘thermogenic’ drugs) such as ephedrine and caffeine which stimulate weight loss by reducing appetite and perhaps by stimulating the body to produce more heat.

Although these drugs offer useful therapeutic effects, there remains a need for more effective obesity treatment drugs. Such a need will fuel tremendous commercial opportunity and so in the future drugs which target gastrointestinal or brain receptors for satiety, or block/mimic the action of satiety altering hormones and substances (such as ghrelin, CCK, PYY, obestatin, leptin, glucagons, neuropeptide Y and the like) might make their way to the market.

Two forms of surgery have been recommended by government consensus panels that can be performed to treat severe obesity. Both are for people with severe cases of obesity, over 100 lbs above ideal body weight (e.g., BMI>40 kg/m²), who have not had effective weight loss with diet, exercise or drugs.

Gastroplasty involves surgically reducing the size of the stomach, thus limiting food intake. Vertical band gastroplasty (VBG) is successful in more than 85% of patients, and weight loss is maintained over prolonged time periods (Barclay Obes Surg. 2004 November-December; 14(10):1415-8). Gastric bypass surgery (e.g. Roux en Y) creates a small stomach pouch and connects this pouch to the second portion of the intestines. Gastric bypass surgery can initially result in substantial weight loss, and approximately 80 percent of patients remain at least 10 percent below their preoperative body weight for 10 years after surgery. The efficacy of the procedure is probably due to the increased sense of fullness with a reduced gastric volume and the symptoms of “dumping” associated with the passage of gastric contents into the intestines, which act as deterrents to eating (Rosenbaum et al. Obesity NEJM Volume 337:396-407 Aug. 7, 1997 Number 6). Although gastric bypass surgery is highly effective, it carries a risk of morbity and it is more extensive and difficult to perform than gastroplasty.

Numerous devices for altering satiety are also known in the art. Some devices restrict stomach size or food intake via bands [e.g. lap band et al. MJA 2005; 183 (6): 310-314] or space occupying elements [e.g. intra-stomach balloons—Obes Surg. 2005 September; 15(8):1161-4]. Others alter stomach or pyloric muscle activity via neuronal or muscular implanted electrodes (Shikora, Journal of gastrointestinal surgery Volume 8, Issue 4, Pages 408-412; Xu et al. Gastroenterology 2005; 128:43-50).

Although numerous treatment approaches are available at present, the most effective approach with the best long term effects is restricted to the treatment of severely obese people and in addition it requires complicated surgery which can lead to severe complications or death.

There is thus a widely recognized need for, and it would be highly advantageous to have, a satiety altering device and method devoid of the above limitations.

### SUMMARY OF THE INVENTION

According to one aspect of the present invention there is provided a gastrointestinal device comprising a band being sized and configured for residing in or around a pyloric sphincter region, the band being for maintaining the pyloric sphincter at a fixed opening size.

According to further features in preferred embodiments of the invention described below, the band is sized and configured for accelerating stomach emptying following ingestion of food.

According to still further features in the described preferred embodiments the band is adapted for implantation between a submucosal layer and a muscle layer of the pyloric sphincter region.

According to still further features in the described preferred embodiments the band is an open band.

According to still further features in the described preferred embodiments the band does not extend into the antrum of the stomach and the duodenum when the device is implanted.

According to still further features in the described preferred embodiments the open band includes at least one end capable of piercing tissue.

According to still further features in the described preferred embodiments the band is composed of at least one material selected from the group consisting of a ceramic material, a polymer, and an alloy.

According to still further features in the described preferred embodiments an internal diameter of the band is selected from a range of 10-25 mm.

According to still further features in the described preferred embodiments the band is configured such that a diameter thereof is adjustable following implantation.

According to still further features in the described preferred embodiments the device further comprises at least one tissue anchoring element attached to the band.

According to still further features in the described preferred embodiments the device further comprises a valve being disposed within the band, the valve being for preventing flow from the duodenum to the stomach.

According to still further features in the described preferred embodiments the device further comprises electrodes being disposed on, or attached to the band.

According to still further features in the described preferred embodiments the band is composed of a plurality of wire helices.

According to still further features in the described preferred embodiments a length of the band is selected from a range of 1-5 cm.

According to still further features in the described preferred embodiments the band is a perforated band.

According to still further features in the described preferred embodiments the band is adapted for implantation between a submucosal layer and a muscle layer of a region flanking the pyloric sphincter.

According to still further features in the described preferred embodiments the gastrointestinal device further comprises structures attached to or integrated with an outer surface of the band, the structures being sized and configured for projecting into submucosal folds of the pyloric sphincter region.

According to still further features in the described preferred embodiments the band is composed of a plurality of interlocking elements.

According to still further features in the described preferred embodiments the band includes a fluid inflatable reservoir.

According to another aspect of the present invention there is provided a method of altering a satiety point of a subject comprising fixing an opening size of a pyloric sphincter of the subject thereby altering the satiety point of the subject.

According to still further features in the described preferred embodiments the fixing is effected by a band being sized and configured for implantation in or around a pyloric sphincter region.

According to still further features in the described preferred embodiments the fixing is effected by implanting a device between a submucosal layer and a muscle layer of the pyloric sphincter region.

According to still further features in the described preferred embodiments the fixing the opening accelerates stomach emptying.

According to still further features in the described preferred embodiments the fixing the opening size of the pyloric sphincter of the subject is effected endoscopically.

According to still further features in the described preferred embodiments the opening size is adjustable following the fixing.

According to yet another aspect of the present invention there is provided method of altering GI functionality of a subject comprising implanting a device between the muscle and submucosal layer of the pyloric sphincter region, the device being capable of increasing a pyloric opening thereby altering GI functionality of the subject.

According to still further features in the described preferred embodiments the implanting is effected by injecting a bio-cement or a biopolymer between the muscle and submucosal layer of the pyloric sphincter.

According to still another aspect of the present invention there is provided a device comprising an element designed and configured for placement in or around a pyloric sphincter region, the device being for initially accelerating stomach emptying following ingestion of food followed by delaying stomach emptying.

According to still further features in the described preferred embodiments the device is capable of shortening the lag phase by at least 50%.

The present invention successfully addresses the shortcomings of the presently known configurations by providing a device and method which can be used to effectively alter satiety using a safe and minimally invasive procedure.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 schematically illustrates the stomach-duodenum junction showing the pyloric antrum (PA), the pyloric canal (PC), the duodenum (D), the pyloric sphincter (PS), the submucosal (SM), mucosal (MC), muscle (M) and serosa (SE) layers and the Pyloric opening (PO).

FIG. 2 illustrates an embodiment of the pyloric band device of the present having a closed band configuration.

FIG. 3 illustrates an embodiment of the pyloric band device of the present invention having circumferential perforations.

FIG. 4 illustrates an embodiment of the pyloric band device of the present invention having circumferential anchors.

FIG. 5 illustrates an embodiment of the pyloric band device of the present invention having an open helical configuration.

FIGS. 6a-b illustrate an embodiment of the pyloric band device of the present invention having a multi-piece configuration.

FIG. 7 illustrates an embodiment of the pyloric band device of the present invention including electrodes.

FIG. 8 is a cross sectional view of a pyloric region with an embedded pyloric band device of the present invention.

FIGS. 9a-b illustrates one embodiment of an indwelling pyloric band device of the present invention.

FIGS. 10a-c illustrates another embodiment of an indwelling pyloric band device of the present invention.

FIG. 11 illustrates an embodiment of an external pyloric band device of the present invention.

FIGS. 12a-b illustrate an external band device (FIG. 12a) which can be dynamically operated to close circumferentially (FIG. 12b) and reduce pylorus opening.

FIGS. 13a-b illustrate the ‘slice and splice’ method of placing the pyloric band of the present invention in between the submucosal (SM) and muscle layers (M) of the pylorus.

FIGS. 14-15 illustrate band implantation into the pylorus using a balloon equipped endoscopic guide.

### DESCRIPTION OF THE PREFERRED EMBODIMENTS

The present invention is of devices and methods which can be used to control pyloric sphincter opening.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

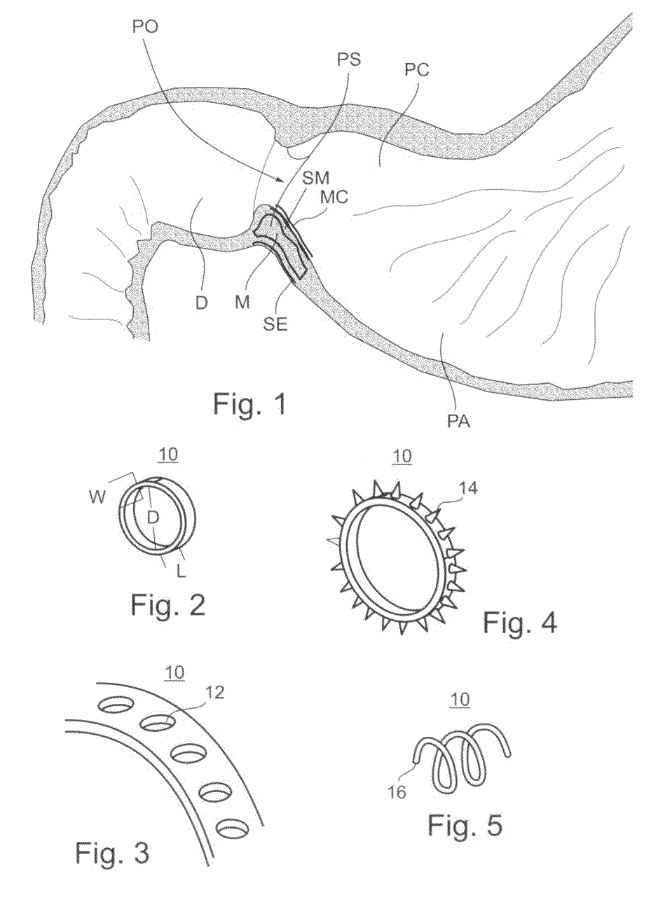

The pylorus is the region of the stomach that connects to the duodenum (FIG. 1). It is divided into two parts: the pyloric antrum (PA, FIG. 1), which connects to the body of the stomach, and the pyloric canal (PC, FIG. 1), which connects to the duodenum (D, FIG. 1). The pyloric sphincter (PS, FIG. 1), or valve, is a ring of smooth muscle (M, FIG. 1) at the end of the pyloric canal which is surrounded by the submucosal (SM, FIG. 1) and mucosal (MC, FIG. 1) layers of the GI tract. The pyloric sphincter is part of a system responsible for controlling the flow of food from the stomach to the duodenum. The pyloric opening (PO, FIG. 1) is the opening surrounded by the lips of the pyloric sphincter (PS) and under certain circumstances also includes part of the pyloric canal (PC). Its diameter varies depending mainly on the degree of contraction and relaxation of the pyloric canal and sphincter. Studies have shown that when fully open, the diameter of the opening can vary between 5-25 mm.

Physiological reflexes in the form of electrical, hormonal, or muscular signals are initiated from the duodenum in response to the presence of an excess of chyme. Such signals are relayed back to other regions of the GI tract to slow or even stop stomach emptying; in addition, satiety-inducing (hormonal or electrical) signals are relayed to the brain (Guyton and Hall Textbook of Medical Physiology, pages 785-6; 2006).

While reducing the present invention to practice, the present inventors postulated that fixing the pyloric sphincter in an open position will induce premature partial gastric emptying and as a result induce a duodenal-activated feedback mechanism which will lead to early satiety and cessation of eating.

Results obtained by prior art studies clearly support the present hypothesis. For example, studies performed by several research groups demonstrated that patients treated for delayed stomach emptying disorders, pyloric closures and other pyloric complications through pyloroplasty or pylorectomy (with or without vagotomy), exhibited weight loss [see for example, Henrion (1981) journal de chirurgie 1981 March; 118(3):155-60.; Castano (2001) Revista Espanola de Enfermedades Digestivas 2001 May; 93(5):315-24.

In addition, numerous studies have shown that bariatric surgeries and procedures in which the pyloric valve is bypassed or removed (e.g. Roux en Y) and partially digested food is passed directly into the small intestine led to weight loss and sustainable changes in eating habits and satiety.

Other studies have shown that satiety can also be effected by a partial closure of the pylorus. For example, patients with pyloric spasms, a condition in which the pylorus is effectively closed, suffer from increased gastric retention times, nausea, vomiting, lack of appetite, and weight loss. Therefore, a pylorus whose opening is too narrow is also effective in causing weight loss and reducing appetite.

Thus, according to one aspect of the present invention there is provided a method of altering a satiety point of a subject. The method is effected by controlling the opening size of a pyloric sphincter region (pyloric sphincter and pyloric canal) of a subject in need. As used herein “a subject in need” is a mammal, preferably a human which could benefit from a controlled pyloric sphincter opening.

Controlling the opening size of a pyloric sphincter region can be effected by fixing the pyloric sphincter and/or canal opening at a fully closed (an opening of 0 mm) or a fully open (an opening of 25 mm or more) position, or at any position in between (i.e. anywhere between 0-25 or more mm).

Several approaches can be used to fix the opening of the pyloric sphincter region.

A number of surgical procedure and instruments have been developed for cutting or ablating tissue such as muscle tissue. Such procedures and instruments can be used to reshape portions of the sphincter ring muscle in order to maintain this muscle in a more open position. Reference is made, for example, to the MediGlobe sphincterotome (http://www.mediglobe.com/).

Other procedures which involve suturing or stapling of muscle tissue or sub-mucosal/mucosal tissue, and/or removal of mucosal-submucosal segments can also be utilized to increase pyloric opening size, see, for example U.S. Pat. No. 5,445,644.

These procedures are similar to pylorectomy/pyloroplasty procedures in as far as the instruments and positioning is concerned, however, pylorectomy/pyloroplasty procedures are designed for increasing flow through the pylorus, while retaining some of the physiological function of the pyloric valve and not for maintaining the pyloric opening at a fixed position as is taught by the present invention.

Fixing pyloric opening can also be effected using a scarring agent, such as, for example ethanol, phenol or acetic acid. Such a scarring agent can be injected into the pylorus muscle while it is in a fully or partially fixed open position to obtain a permanent effect of a non-constrictive muscle ring with a fixed pyloric opening. Other methods which can be used to prevent the pylorus muscle from contracting are local applications of heat, radio frequency, ultrasound energy, laser or physical cutting. Preferably, these approaches are applied directly to the muscle tissue in order to avoid injuring the submucosal and mucosal layers of the pyloric region. Therefore, the current invention also envisages devices designed capable of delivering such scarring or muscle-inactivation energy to the pyloric muscle without damaging to a significant extent the mucosal and submucosal layers. Exemplary devices include an inflatable assembly or catheter with a multi-pronged needle injector or slender energy-transfer needles that do not damage the submucosa and mucosal layers while transferring the selected agent or energy directly to the pyloric muscle layer.

In a further embodiment, a muscle paralysis agents, for example botulinum toxin (Botox), or a muscle relaxing agent, for example nitric oxide [see, Allescher, Am J. Physiol. 1992 April; 262(4 Pt 1):G695-702], can be injected, released over time (e.g. through injection of a slow release formulation such as that described in, for example, U.S. Pat. No. 6,506,399) or generated in the vicinity of the pylorus muscle to obtain a temporary or long-lasting effect of a non-constrictive muscle ring with a fixed pyloric opening. Injection means, time release drug delivery polymers or drug delivery pumps in which such agents can be delivered or generated are well known in the art.

The effect of one-time injections of Botox into the pyloric sphincter to improve gastroparesis symptoms is known in the art (Lacy, B. et. al., Diabetes Care, volume 27, number 10, October 2004 pp 2341-7). Likewise it has been shown that injection of Botox into the pyloric and antrum regions of the rat and human stomachs caused weight loss (Coskun H, Obes Surg. 2005 September; 15(8):1137-43, and Rollnik J D, Annals of Internal Medicine Volume 138 Number 4. February 2003: pp. 359-360) thereby substantiating the satiety feedback mechanisms proposed by the present inventors.

Preferably, the pyloric sphincter region is fixed at an open position such that it is incapable of fully closing. As is mentioned hereinabove, such fixing is advantageous in that it enables premature gastric emptying and/or overloads the duodenum with chyme which in turn triggers a duodenal activated satiety mechanism.

Although the above described procedures can be utilized to fix open the opening of the pyloric sphincter, such procedures are cumbersome to perform and may not be totally effective in maintaining the pyloric opening at a fixed predetermined diameter.

Thus, the method of this aspect of the present invention is preferably practiced using a dedicated device which enables to accurately and fully control opening diameter of the pyloric sphincter region (preferably the pyloric aperture) through a laparoscopic or endoscopic procedure.

Such a device can be adapted for use in (indwelling or implanted) or around (adjacent to the serosa) the pyloric sphincter region. Preferably, such a device is implanted in, or positioned at, the pyloric sphincter or it is implanted or positioned immediately adjacent to the pyloric sphincter (e.g. positioned at the stomach or duodenal side of the pyloric sphincter or flanking the sphincter from both sides). Application of the present invention can be combined with a vagotomy for enhanced procedural outcomes when needed.

Referring now to the drawings, FIGS. 2-8 illustrate several embodiments of a device suitable for fixing the opening of the pyloric sphincter in accordance with the teachings of the present invention, which device is referred to herein as band 10.

As used herein, the term “band” refers to any open or closed structure having a substantially circular cross shape, including, but not limited to, a cylinder (eg a short tube), a taurus, a coil and the like, the band can be rigid, semi-rigid or elastic in nature. The band can have a fixed or dynamic inner (opening) diameter as is further described hereinunder.

As is further described hereinunder, band 10 is sized and configured to be placed in or around the pyloric sphincter. Thus, band 10 can reside within the pyloric ring muscle, between the muscle and submucosa or submucosa and mucosa, in the opening juxtaposed against the mucosa or outside the pyloric sphincter region of the GI tract (i.e. around the stomach exterior).

Numerous sizes and configurations of band 10 are contemplated herein. FIG. 2 illustrates a simple configuration of band 10 having a length L, a width W and a diameter D. Length of band 10 can vary from 1 to 50 mm. The width of band 10 is selected from a range of 0.1 to 10 mm. It will be appreciated that selection of appropriate sizes and configurations depends on the material from which the band is made, the tissue region of placement as noted above and the degree of pyloric opening desired.

The diameter of band 10 is also selected according to the pyloric opening desired and the position of band 10 (in or around tissue). The desired opening diameter is selected according to the flow desired through the pyloric canal and pyloric sphincter.

In normal individuals, the pylorus restricts food particles larger than 1 mm from passing to the duodenum (Pera et al., J Dent Res 81(3):179-181, 2002) and as a result, stomach emptying initiates approximately 20-50 minutes following ingestion of food (Kasicka-Jonderko et al. World J Gastroenterol 2006 Feb. 28; 12(8):1243-1248;).

In order to decrease this lag time in stomach emptying following ingestion and thereby induce early satiety, the pyloric opening diameter (as determined by band 10) is preferably selected such that it enables flow of food particles larger than 1 mm, preferably, larger than 2-5 mm through the pylorus. Thus, band 10 of the present invention can be selected of a diameter which maintains the pylorus fully or partially open and as a result decrease the lag time following food ingestion to 15 minutes, preferably 10 minutes more preferably 5 minutes or less.

As is mentioned hereinabove, band 10 can also be selected of a diameter which maintains the pylorus in a partially open position.

The pylorus opening is about 9 mm in diameter when the pylorus muscle is relaxed (Keet et al., The Pyloric Sphincter Cylinder in Health and Disease, online edition, chapter 11 page 44). A rigid band 10 that maintains the pylorus at a partially open position (e.g. about 2-7 mm, preferably 5 mm) can decrease the lag time in stomach emptying (as described above) and at the same time constrain the maximum amount of chyme transferred to the duodenum by preventing the pylorus from fully opening. By delaying the maximal rate of stomach emptying, a second satiety feedback mechanism is therefore activated. In this embodiment, band 10 can be anchored or sutured to the muscle thus preventing it from relaxing, it can be placed such that it enables the muscle to relax and physically separate from band 10 while band 10 maintains pressure upon the submucosa and constrains it from retracting along with the smooth muscle layer, or band 10 can be configured having an elastic outer layer and a rigid inner layer such that an outward pull of the muscle elastically deforms the outer layer and yet maintains the rigid inner layer in contact with and constraining the submucosa.

Therefore, a rigid band 10, properly sized, will provide both the benefit of increasing the minimum size of the pylorus opening to allow chyme to prematurely enter the duodenum while also limiting the maximum gastric emptying rate by not allowing the pylorus to fully open.

It will be appreciated that in cases where one does not wish to constrain the maximum pyloric opening size, band 10 can be attached to, and expand along with, the muscle layer of the pyloric sphincter but resist compression and therefore maintain a minimum opening size. Such properties can be designed into the material of the pyloric band, or be a function of the mechanical design of the band itself. Such a configuration can be realized using a material in band 10 which is responsive to force/motion (e.g. thicksotropic polymer), such that compression of the pyloric muscle stiffens band 10.

Band 10 can be fabricated from one or more materials suitable for implantation in a body. Examples of suitable material include polymers such as polyurethane and polypropylene, Silicone™, Teflon™, ceramics, NITINOL, passive metals, alloys and the like.

Preferably, the material selected is biocompatible or includes a biocompatible coating.

Additional coatings for preventing biofilm formation, encapsulation, erosion and antigenic reactions can also be employed. The prior art is replete with examples of materials that can be used for such purposes [see for example, Baveja et al. Biomaterials. 2004 September; 25(20):5003-12].

Coatings including medicaments or pharmaceutically active agents such as muscle relaxants, Botox and the like are also contemplated herein.

In cases where implantation of band 10 is temporary (further described below), use of biodegradable or bioresorbable material is also contemplated herein. Examples of such material can be found in www.sigmaaldrich.com/Area_of_Interest/Chemistry/Materials_Science/BiocompatibleBiodegradable.html.

Band 10 can be fabricated from one or more pieces each fabricated using well known techniques such as casting injection molding, extrusion and the like. One of ordinary skill in the art would be more than capable of fabricating band 10 using such techniques.

Band 10 can be rigid or semi rigid (e.g. elastic) depending on its intended purpose and point of placement.

Width of band 10 depends on the site of placement and the hardness or rigidity desired.

In order to maintain the pylorus open, the maximum pressure in pyloric sphincter that band 10 needs to resist is typically 34 mm Hg (0.65 psi) (AD Keet, Pyloric textbook chapter 13, page 51). From the same text, average pylorus aperture diameter is 8.7 mm in motor quiet phase with a width of the sphincter being 4.7 mm. Therefore assuming that the submucosa is 2.5 mm thick, and that the desired fixed internal pyloric opening diameter is, for example, 5 mm, a band 10 being 10 mm (0.4″) in diameter and 5 mm (0.2″) in width, and having a cross sectional area (A) of 50 sq mm (0.08 sq inches) can be used to maintain the pylorus at a fixed position with an opening 5 mm in diameter.

The maximum force on such a band 10 configuration can be calculated using the following formula:

F=P×A/2

Wherein P=Max pressure in sphincter (0.65 psi) and A=cross sectional area (0.08 sq inches).

Using the formula above indicates that such a band needs to resist a maximal compressive force (F) of only 12 grams (0.026 pounds) to keep the sphincter open with a 5 mm internal aperture. Thus, a very thin ring of rigid, semi rigid or even flexible material such as NITINOL can be used as band 10 without fear of it buckling or deforming.

Furthermore, band 10 can have a known elasticity designed into it, either through mechanical, geometrical or material properties of the band, in order to transfer a desired biasing force to the sphincter. For example, band 10 can keep the pyloric opening at a partially open state by simply applying an outward radial force on the pyloric sphincter, where the force exerted by band 10 could decrease, either linearly or non-linearly based on Hooke's law of spring force as a function of displacement and spring constants, as the pylorus opens naturally. In quantitative terms, if band 10 as described in the preceding paragraphs provides the equivalent of 6 grams circumferential expansion force, such a force would neutralize approximately 0.32 psi of the sphincter closure pressure and effectively weaken the sphincter's strength by a factor of 2. Thus, band 10 can act as a biasing force to either help open or close the pylorus in order to attenuate or augment the natural sphincter function.

When implanted between tissue layers (e.g. between the ring muscle and the submucosa), band 10 is fabricated having a width which is preferably 5 mm or less so as to minimize separation between the submucosa and muscle. Minimizing tissue separation will ensure rapid healing and maximize tissue layer adhesion around implanted band 10. Connective tissue holding the submucosa to the pyloric muscle may also act to keep band 10 anchored longitudinally in the sphincter throughout the various phases of pyloric motility.

FIG. 3 illustrates band 10 having circumferential perforations 12 (e.g. holes). Perforations 12 further facilitate tissue healing in an implanted band 10 or serve as suture or staple anchors in configurations of band 10 which are positioned in or around the sphincter. FIG. 4 illustrates band 10 having circumferential anchors 14 which function in anchoring band 10 against the mucosa or the ring muscle. Numerous configurations of anchors 14 are contemplated herein, including screws, spikes (shown in FIG. 4), hooks, tissue adhesives, barbs, tacks, clips, sutures, staples, attachments strips, loops and the like. Such anchors can be deployable using springs, shape memory alloy segments and the like. Numerous tissue anchor configurations are known in the art and so no further description of such anchors is necessary herein. Further detail of various attachment/placement approaches for band 10 is provided hereinbelow and in the Examples section which follows.

Band 10 can be a closed band (as is exemplified by FIG. 2) or it can be an open band (e.g. a simple open band). FIG. 5 illustrates a helical (open) configuration of band 10. Any number of helices is contemplated herein (2.5 helices shown in FIG. 5) depending on band 10 position and function. An implanted helical configuration can also include a sharp tissue piercing or blunt tissue separating end 16 which can be used for implanting band 10 within tissue through a spiraling—tissue boring action (cork screw). Alternatively, a rigid helical boring tool can first be rotated from within the GI tract into the tissue and then removed to create a channel through which a less rigid helical band of similar diameter and pitch can then be inserted using a similar rotary motion.

Likewise, an open band can be inserted after a tissue separation/insertion tool has from within the GI tract pierced the mucosal and submucosal layers in one or more points, and in a rotary motion separated the submucosal and muscle layers in preparation for an open band being inserted between these layers, also in a rotary motion from within the GI tract.

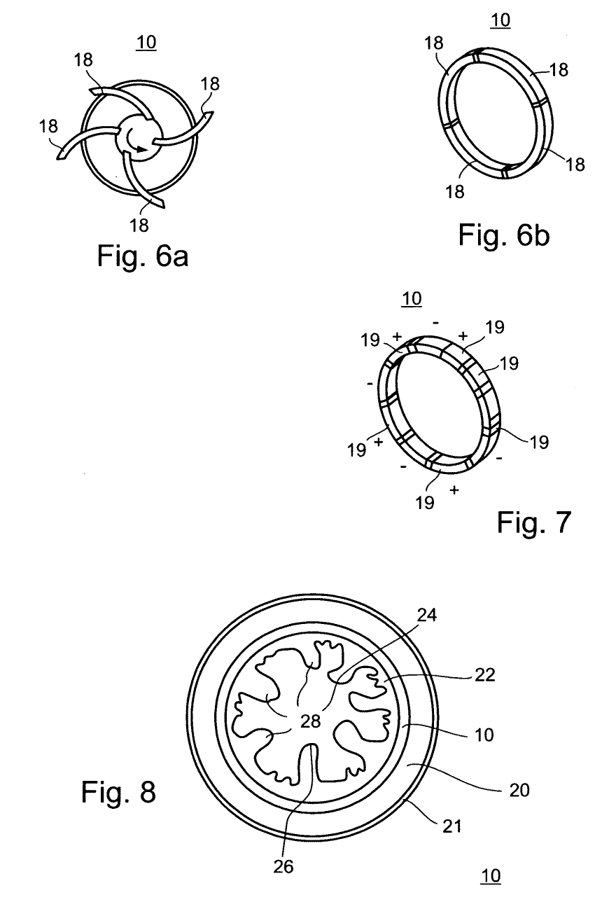

FIGS. 6a-b illustrate band 10 which is fabricated as a plurality of separately positionable/implantable elements 18 (four shown). Preferably elements 18 are configured having interlocking ends, such that following positioning thereof, the ends are interlocked to from a rigid band. As is further detailed hereinunder with respect to positioning of band 10, such a configuration can be positioned via minimal tissue perforation from within or outside of the GI tract.

Band 10 can also include a valve device for reducing or eliminating backflow from the duodenum to the stomach. Such a valve can be, for example, a single flap or a bicuspid, tricuspid, or a higher number configuration of flaps disposed within band 10. Preferably such flaps are formed from a relatively flexible material such as silicone. Other valve configurations can include spring loaded “trap doors”.

One preferred valve configuration is described hereinbelow with respect to FIG. 8.

Band 10 can also include electrode surfaces or attached electrodes which can connect to the band and be extended to electrically stimulate adjacent tissues such as branches of the vagal nerve, the enteric nervous system, or gastric, antrum, and/or duodenal tissues either from within the muscle layer, outside the serosa, or interior to the mucosal surfaces. FIG. 7 illustrates band 10 with surface mounted electrodes 19. Additional or alternative electrodes can be used for sensing of muscle activity in which case, information sensed thereby can be used to control, for example, the diameter of band 10 (in the case of the adjustable configuration of band 10 described below) or to induce or control function of other GI devices such as space altering (e.g. gastric bands) or space occupying (e.g. intra-stomach balloons) devices communicating therewith. Communication between electrodes of band 10 and any GI implanted devices can be effected using wireless communication or implanted wires which can be implanted between the submucosa and muscle layers of the GI tract.

Prior art studies have shown that while the exact mechanisms of gastric stimulation remain incompletely understood, it appears that the implantation of an intra-gastric stimulator (IGS) is associated with weight loss, an improvement (decrease) in blood pressure in hypertensive patients, and a reduction or elimination of symptoms in those who had GERD. (Cigaina Obes Surg. 2004 September; 14 Suppl 1:S14-22). More over Xiaohong et al. (Gastroenterology 2005; 128:43-50) claimed that “PES (pyloric electrical stimulation) with long pulses significantly delayed gastric emptying, impaired gastric myoelectrical activity, inhibited antral contraction, and reduced food intake without inducing any noticeable symptoms in dogs”.

Thus, band 10 of the present invention provided with electrodes can be used to fix open the pyloric valve and/or stimulate (continuously or at intervals) the adjacent tissue (preferable gastric/duodenal). In an alternative embodiment, the pyloric band can be an open or closed fully-flexible carrier of the electrodes and not control normal pyloric motion or gastric functioning through mechanical means, but rather through electrical means alone. An advantage of positioning the electrodes directly on a band implanted between the submucosa and muscle layers of the pylorus is that the electrodes are directly in electrical communication with the muscle and nerve endings, and therefore the difficulties associated with penetrating and anchoring one or more electrodes through the submucosa or serosa layers are eliminated. Power for the electrodes can be provided from internal energy storage means, such as a battery or capacitor that are recharged through a power source outside the body (see for example U.S. Pat. No. 6,061,596). Likewise the electrodes can be controlled using commands delivered from an attached processor and/or electronic circuitry, or from a control unit remaining outside the body via wireless communication as known in the art (e.g. U.S. Pat. No. 6,061,596). The triggering of electrical stimulation could be due to sensing a change in a pyloric parameter such as motion, electromyograph (EMG) or muscle tone with sensors built into the stimulator.

Band 10 can also be constructed such that a configuration thereof can be modified following implantation. For example, band 10 can include hinged regions fabricated from a shape memory alloy (e.g. NITINOL) which when activated (via applied energy, such as electricity, RF etc) would modify a shape of band 10 (e.g. from circular to linear). Such a mechanism can be used to control (via an implanted or a remote controller) the size of the pyloric sphincter opening and when desired used to completely close the pyloric opening. Devices using shape memory alloy to control for fecal incontinence include, for example, a publication by Luo Y. et. al, Smart Mater. Struct. 14 (2005) 29-35. One skilled in the art could adapt such technology for use with this invention.

Band 10 can include one or more fluid inflatable lumens that can be used to decrease the inner diameter of band 10 and thereby force the sphincter opening to close. Such a configuration of band 10 can be implanted in or around the pylorus or around the serosa. In the latter case, band 10 is similar in operation to a gastric band with the exception that in the non-inflated state, band 10 of the present invention maintains the pyloric sphincter open via a radial pulling force. The opening and closing of the pyloric sphincter, as effected by the inflation and deflation of the inner lumen of band 10 could be a dynamic process, thereby taking over the function of the pyloric sphincter in a manner optimized to alleviate the patient's GI problem or eating disorder.

An alternative configuration of an adjustable external band 10 can include two or more longitudinally spaced interconnected rings which can be displaced with respect to each other along the serosa outside of the pylorus region. In such a configuration, the rings of band 10 can be displaced longitudinally towards (or away from) each other to thereby ‘pinch’ tissue positioned therebetween and thus in effect pull the pylorus tissue outward and as a result open (or close) the pyloric valve or canal.

Devices with inflatable lumens for occluding bodily passageways are known in the art, see for example American Medical Systems Acticon Neosphincter used for fecal incontinence. Numerous systems for delivering energy to an intrabody-positioned device for the purpose of mechanical lumen constriction are known in the art, see for example, U.S. Pat. No. 6,471,635 used for fecal incontinence. One of ordinary skill in the art can readily adapt such systems for use with the present invention.

As is mentioned hereinabove, band 10 of the present invention can be configured for placement in or around the pyloric region. Since the pylorus is a highly active sphincter moving in both radial and longitudinal directions, it is presently preferred that positioning of band 10 is effected in a manner which minimizes stress on the pylorus tissue and adjacent regions and yet enables fixation of the pyloric opening at a predetermined and effective diameter.

Thus, one presently preferred configuration of band 10 is an intra-pyloric band which resides between the submucosa and ring muscle layers of the pylorus in a region that is either around the pyloric aperture or immediately adjacent thereto. As is shown in FIG. 8 in such a preferred configuration, a band 10 residing between ring muscle 20 and submucosal 22 layers of the pyloric region of the GI tract can maintain pyloric opening 24 at a fixed position (open in FIG. 8) by resisting contraction of the ring muscle of approximately 34 mm Hg. Although submucosa 22 and mucosa 26 form ‘flaps’ (indicated by 28) which extend into opening 24, these flaps are flaccid and thus do not substantially influence passage through opening 24. Such flaps can be further extended into the opening space via static or dynamic projections disposed inwardly from band 10. Such projections can be fabricated from a relatively soft material (e.g. silicone) which would provide enough rigidity and area coverage to the flaps formed from mucosa 26 and submucosa 22 such that they in effect form a one way valve which resists backflow of bile acid from the duodenum and yet do not obstruct flow from the stomach to the duodenum. The flaps of band 10 can also be directed to expand or contract on demand (e.g. via a fluid filling mechanism, mechanical protrusions, artificial muscle, or polymer swelling) using a power source and commands issued through wired or wireless means known in the art, or in response to an environmental condition (e.g. pressure on the sphincter, motion of a part of the gastrointestinal (GI) tract, motion of ingested material through the GI tract, changes in pH of a region of the GI, mastication, or by communicating with an separate device that signals the beginning of an eating event, etc).

It will be appreciated that the above described configuration of band 10 (as well as the indwelling configuration described in greater detail below), can also be used to hyperextend ring muscle 20 and thus increase opening 24 opening beyond the physiological range.

Since in this configuration, band 10 is fixed in the connective tissue between the submucosa and muscle layers it may not require additional anchoring or suturing into tissue layers, although muscle anchoring can be employed for insuring that band 10 does not migrate.

The above described configuration offers several distinct advantages:

(i) it is implanted outside the mucosal layers and so it is not exposed to stomach or bile acids and thus does not necessitate erosion protecting coatings, nor does it cause the formation of ulcers in the submucosal or muscle layers through leakage of stomach acids into these regions because there is no chronic piercing of the mucosal layer;

(ii) it is secured between tissue layers and so it is less prone to migration; and

(iii) it does not cover the surface area of the mucosa in the pyloric region this feature may be of importance for hormonal regulation of physiological stomach motility since it has been shown that the pyloric mucosa is covered with receptors for the CCK hormone which may play a role in stomach motility and gastric emptying (Science 2005 in the article “the gut and energy balance: visceral allies in the obesity wars”).

As is mentioned hereinabove, the present invention also envisages indwelling and external configurations of band 10.

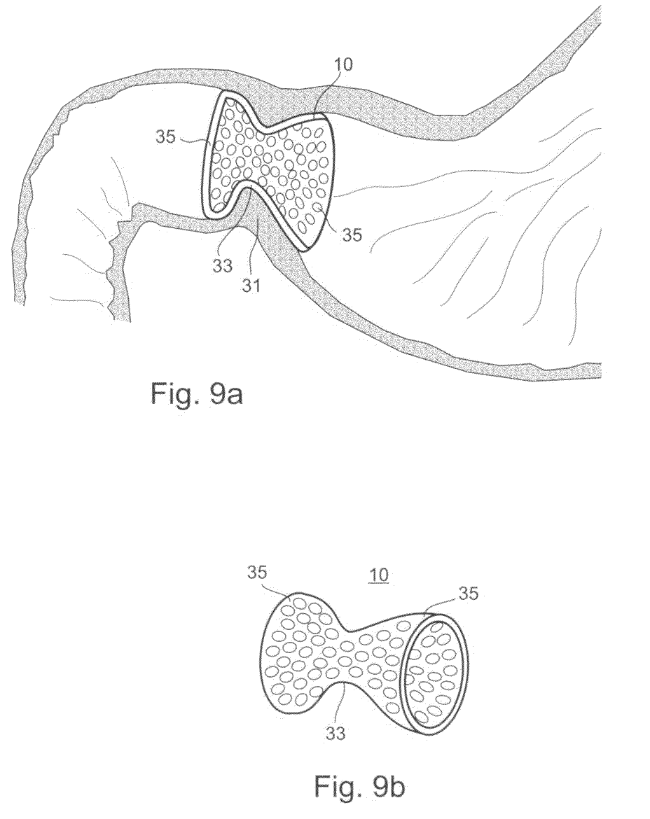

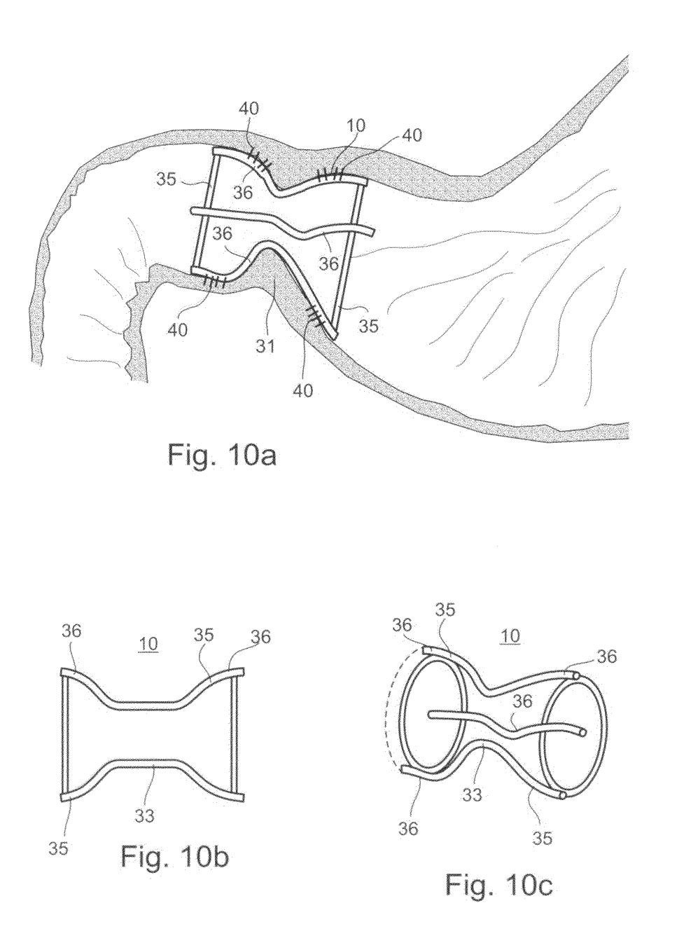

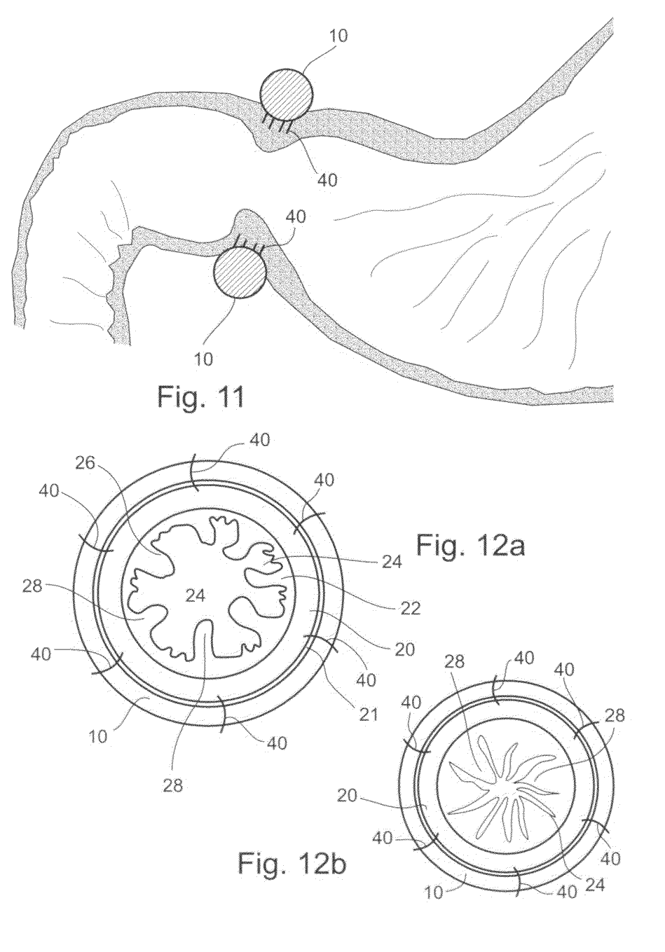

FIGS. 9a-11 illustrate several exemplary embodiments of indwelling (FIGS. 9a-10c) and external (FIG. 11) configurations of band 10.

FIGS. 9a-b illustrates a porous funnel like configuration of band 10 which is designed for placement within the opening of pyloric sphincter 31. This indwelling configuration of band 10 is shaped as an hourglass, with a tapered portion 33 residing in the opening and ends 35 flanking the opening; this shape is employed in order to minimize migration of band 10 under pyloric movements.

Band 10 illustrated in FIGS. 9a-b can be fabricated from a polymer or alloy and optionally also provided with anchors or suture holds.

Typical dimensions for such a band 10 configuration are of length 25-50 mm, external edge diameters of 25-50 mm and a narrow sphincter region diameter of 2-15 mm.

FIGS. 10a-c illustrate a configuration similar to that shown in FIGS. 9a-b, although in this case, the funnel-like shape is formed from interconnected struts 36.

In both the above described configurations, band 10 is constructed to be rigid enough to resists contractions of the ring muscle of the pylorus.

FIG. 11 illustrates placement of an external band 10. It will be appreciated that this configuration requires tissue anchoring in order to maintain the pyloric sphincter opening in a fixed (preferably open) position. Such anchoring can be effected via permanent or degradable sutures 40, clips, or the use of tissue anchors.

FIG. 12a illustrates an external band where band 10 is fixed via sutures 40 to the serosa 21 and muscle layers 20 of the pylorus. Band 10 in one state can maintain pylorus opening 24 patent by applying the required radial tension forces on pylorus muscle 20 through sutures 40 or anchoring mechanism. An external static band is sufficient to cause premature emptying of chyme into the duodenum and initiate satiety (mucosa 26 and submucosa 22 are also shown).

FIG. 12b illustrates a further feature of the invention where band 10 can be dynamically operated to close circumferentially and close pylorus opening 24 by the compression of flaps 28 thereby overriding, replacing, enhancing or augmenting the normal closing action of the pylorus, either with or without regard to the normal nerve signals to the pylorus. Therefore, in this embodiment, band 10 would be capable of keeping the pylorus open in one extreme state and closing the pylorus altogether in the other extreme state in a static or dynamic fashion. Reference is made to U.S. Pat. No. 6,471,635 for technology that one skilled in the art could use to construct such a system. Various mechanical actuators, shape memory alloys, artificial muscles (see, for example Madden J D, Science Vol. 311, 17 Mar. 2006 or Scientific American October 2003), ratchet and pawl mechanisms, worm gear drives, and other means to shorten or lengthen the circumferential or radial dimensions of band 10 are known in the art and can be adapted by the present invention. In a further embodiment, the internal part of band 10 is inflated with a fluid (gas or liquid) either from a reservoir within the body or from a transcutaneous saline injection in a manner similar to a gastroesophageal laparoscopic band (“lap band”) to adjustably set a static and fixed pyloric opening.

In another embodiment to induce satiety, for example, the pylorus could be kept open or even opened beyond its physiological state at the beginning of a meal to cause a decrease in the lag time of gastric emptying of chyme into the duodenum to initiate the satiety feedback loops discussed earlier, and thereafter partially close the pylorus to cause the additional ingested food to distend the stomach and therefore further induce satiety, through a separate and perhaps independent and additive mechanism. Thus this embodiment of the invention provides full control of the pyloric function.

Band 10 in FIG. 8 can be sized to keep the pylorus in a neutral or slightly dilated resting state when no power is applied to the device. When power is applied to the device, band 10 can be driven to grow in circumference in order to expand the pylorus opening, and band 10 can also be driven to shrink in circumference to close the pylorus opening in a dynamic manner. Therefore, band 10 can be designed to have a fail-safe power-off setting which also helps to minimize erosion into the surrounding tissues due to the low forces involved in the resting state.

Supplying power and control signals to the actuators in band 10 can be through wired or wireless means known in the art, or in response to an environmental condition (e.g. pressure on the sphincter, motion of a part of the gastrointestinal (GI) tract, changes in pH of a region of the GI, beginning of eating signals).

With further reference to FIGS. 12a-b, band 10 can be made from a shape memory alloy. Band 10 can be of rigid construction where in the passive state maintains the pylorus open by applying radial tension on the pylorus serosa 21 and/or muscle 20. The compressive forces to close pyloric opening 24 can be provided via a mechanical impingement on serosa 21 surface (e.g. via a fluid filling mechanism, mechanical protrusions interspersed between sutures 40 or anchors, polymer swelling, etc). In a further embodiment, the regions of the tissue anchors or sutures 40 can move radially with reference to a rigid external band 10 and thereby provide tension forces on pylorus muscle 20 to open pylorus opening 24 or compressive forces on pylorus muscle 20 to close the pylorus opening 24. In a further embodiment, rigid band 10 can be divided into one or more hinged segments that flatten out to close the pylorus opening 24 or open up into a circular cross section to open pylorus opening 24.

Several approaches can be used to position band 10 within, around or in the pyloric sphincter.

Trocar-introduced laparoscopic instruments such as the methods commonly used to insert a gastric “lap band” can be used to position band 10 around the pyloric sphincter region in contact with the stomach serosa. Manual suturing or semi-automated suturing devices such as the Bard EndoCinch can be used to staple or suture band 10 in place.

Transgastric approaches (for example as described in U.S. Pat. No. 6,572,629) can also be used where the insertion device is inserted into a gastric endoscope and cuts through the pyloric region to place band 10 around the outside of the pyloric sphincter region. The subsequent incision in the gastric wall is then sealed and left to heal.

Placement of the indwelling and implanted configurations of band 10 can be effected using an endoscope mounted guide. Examples of technologies and systems used to position, insert and seal around a device implanted in the pyloric region using endoscopic means are described in U.S. patent application 2004/0019388 which is incorporated herein as reference, including for the purpose of providing additional details to the Examples section below.

It will be appreciated that in cases where band 10 includes electrodes which communicate with stomach or duodenal implanted devices, band 10 and other components of such a system can be implanted through an incision made in the antral region of the stomach.

The Examples section below described one suitable guide system and use thereof in positioning the intra-sphincteric band of the present invention.

The devices and methods of the present invention can be used for treating a variety of conditions and disorders which are associated with satiety. As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Conditions and disorders associated with satiety include, but are not limited to, obesity and obesity related disorders such as for example anorexia and bulimia. Furthermore, a pyloric band could precondition patients that are candidates for bariatric surgery as a simple way for weight reduction prior to surgery, and by providing an adjustment period for managing dumping syndrome symptoms.

Example individuals who may benefit from the pyloric ring for conditions other than eating disorders or obesity are described below.

Gastroparesis is abnormal functioning of the stomach without any physical evidence of obstruction, a debilitating condition which is mainly a complication of diabetes. Other etiologies include: (a) Parkinson and other neurological conditions (b) post vagotomy with pyloroplasty and other gastric surgeries (c) immune diseases such as lupus and scleroderma; and (d) gastric scaring due to past ulcers. Current treatments of these conditions vary from extensive life style and diet modification through pro kinetic medications and electrical stimulation. A pyloric band fixing the pylorus at or around its normal opening size would serve to accelerate gastric emptying in these patients and significantly ameliorate the symptoms of the disorder.

Gastroesophageal reflux disease (GERD) is a common condition; current treatment is acid lowering medications and surgery. GERD patients may benefit from a more open pyloric sphincter through earlier and quicker gastric acid emptying or reduced intra-gastric pressure.

Peptic ulcer disease (PUD) is an ulcer occurring near the pylorus that may cause strictures as a result of the inflammation. These patients after eradicating the cause of the ulcer may benefit from a device that keeps the otherwise narrowed pylorus open.

Post major abdominal surgical patients often complain of delayed gastric emptying symptoms. They may benefit from an open pyloric sphincter that keeps the flow of gastric secretions and food. In such patients, the need for the band may be temporary, and therefore the ability of removing the ring or having it degrade and or bio-absorb over time would be beneficial.

Hypertrophic pyloric stenosis (HPS) patients suffer from mechanical gastric outlet obstruction, and may benefit from a method keeping the pylorus open.

The present invention further encompasses a method of controlling and adjusting the proper settings for the present device, as well as the optimization of the parameters controlling the dynamics of the pyloric opening and closing device to best match individual patient needs.

An example program for weight loss could comprise detecting the beginning of a meal based on sensing stomach motility electrically or mechanically, opening the pylorus to allow chyme to prematurely enter the duodenum with a decreased lag time thereby creating a first feedback signal of satiety, then after a few minutes closing the pylorus which causes any further ingested food to distend the stomach, thereby causing a second feedback signal of satiety. Once ingestion has stopped, the pylorus would then be cycled in a physiologically normal manner to allow food into the duodenum, but only after the patient has stopped eating. Such a “pyloric opening and closing regime” could consist of personalized parameters including, but not limited to, opening and closing diameters of the pylorus opening, response time between actions, sensitivity to input GI data to the device, desired pylorus sphincter pressure, duration and speed of pyloric sphincter opening and closing actions, can be stored on board a memory chip in the device or in an external controller and adjusted from time to time based on objective data such as GI functioning or desired body mass, or based on patient preferences or doctor input.

It will be appreciated that a band 10 configuration which functions in fixing open the pyloric valve region can also be realized via use of an injectable adhesive or space filling cement. For example, a biocompatible-cement (see, for example, U.S. Pat. No. 4,804,691) or a biocompatible-polymer adhesive [e.g. Mo et al., J Biomater Sci Polym Ed. 2000; 11(4):341-51] can be injected between the submucosa and muscle layers of the pyloric region and allowed to cure while the pyloric sphincter is maintained in an open position (via for example, a mandrel). Once the cement or polymer sets and hardens it can either form band 10 which is capable of maintaining the pyloric sphincter open, or it can fix the submucosal and muscle layers thereby partially or fully restricting pylorus muscle function, or it can form band 10 as a flexible spring element which enhances or restricts pyloric sphincter muscle movement. Injection of a cement or adhesive can be effected via multiple injection sites or through a single injection site. The latter approach is preferably effected using a guide for guiding the injected material around the pyloric sphincter muscle to form an adhesive or space filling band.

Although use of the pyloric band device is presently preferred for treatment of the above described disorders, the present invention also envisages alternative devices which can be implanted between the submucosal and mucosa of the pyloric sphincter region of the GI tract. For example, such an implanted device could function as a platform for drug or hormone release or as a carrier of electrodes for neurostimulation.

It will be appreciated that since side effects of having a pyloric sphincter open for a prolonged period of time can include dumping syndrome and gastritis, treated individuals can be further treated for such side effects via behavioral and pharmaceutical interventions (see for example http://emedicine.com sections on Gastritis and Peptic Ulcer Disease and Dumping Syndrome). Furthermore, proof that a patient can live chronically with undigested or partially digested large food particles in their small intestine is provided by patients adapting successfully to Roux en Y gastric bypass surgery, in which a small stomach pouch is connected directly to the small intestine, altogether bypassing the pylorus.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Example 1

### Sub-Mucosal Implantation of a Closed Pyloric Band

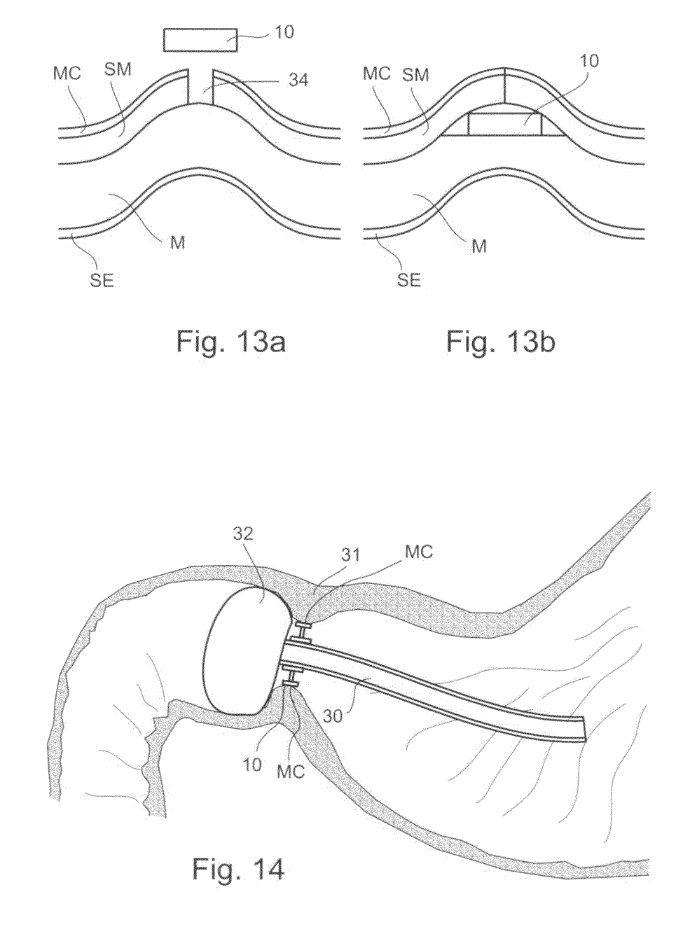

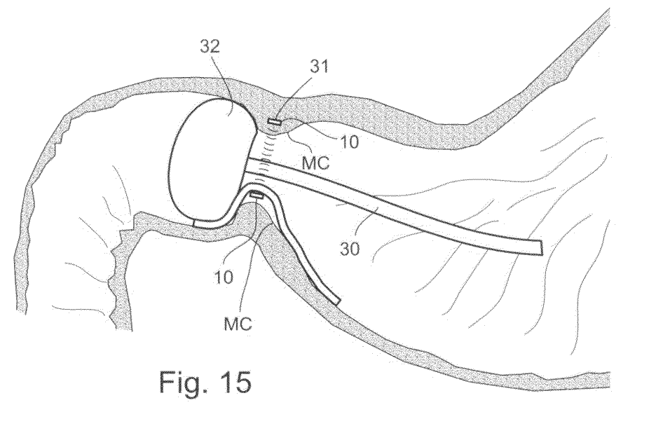

FIGS. 13a-15 illustrate a procedure (FIGS. 13a-b) and a guide (FIGS. 14-15) suitable for placement of an implanted configuration of band 10 of the present invention.

Although numerous approaches are contemplated herein, one approach, which is termed herein, ‘slice and splice’ is preferred for its simplicity and safety.

A guide 30 (FIGS. 14-15) is inserted through an endoscope and anchored against the distal end of the pyloric sphincter 31 using an inflatable balloon 32. Alternative methods known in the art for determining the position of the guide include direct visual feedback, fluoroscopic guidance, and potential or pH differences between the pyloric antrum and the duodenum. The guide can be advanced through the working channel of a GI endoscope.

Optionally, a dilator or mandrel can be advanced over the guide to expand the pyloric sphincter to a desired diameter. Band 10 is then advanced over the guide to the site of implantation and positioned against the mucosa (MC) at the site of implantation. As is shown in FIGS. 13a-b, a small region (2-5 mm) of the mucosal/submucosal layer is sliced open (indicated by 34) to gain access to the muscle layer (M). Band 10 is partially inserted through the slit and the slit is sutured, stapled, clipped or welded closed. This procedure is then repeated several times along the inner circumference of the pyloric valve until band 10 is entirely implanted between the pyloric sphincter ring muscle and the submucosal layers. It will be appreciated that when needed, additional suturing or stapling of band 10 to the ring muscle layer can be effected during the above described procedure in order to further anchor band 10 in position.

Following implantation, balloon 32 is deflated and guide 30 and endoscope are retracted.

### Example 2

### Removal of the Closed Pyloric Band

When the desired effect is obtained or a decision is made to remove the pyloric band, an endoscope is inserted into the stomach of the patient and the pyloric sphincter region located with any of the means described above. The surgeon then clips the ring in one or more locations through the submucosa. Using forceps the surgeon slides out segments of the cut ring through the incision in the submucosa. The incisions in the submucosa either self-heal or are sutured, stapled, welded, or clipped together. Normal pyloric function is therefore restored.

### Example 3

### Sub-Mucosal Implantation of an Open Pyloric Band

As in example 1, an open or helical rigid band is delivered to a dilated pyloric sphincter and inserted through a single incision in the submucosa and rotated into place where the blunt leading edge of the open band separates the tissues and excavates a space between the submucosa and muscle layers. The band can be sutured in place to prevent longitudinal migration using degradable sutures around the submucosa, the band and the smooth muscle. Alternatively, the connective tissue between the submucosa and the muscle will restrain the band from moving relative to the sphincter muscle. The incision in the submucosa either self-heals or is sutured, stapled, welded, or clipped together with, for example a Boston Scientific Resolution™ clip. The net result is a pylorus opening that remains open during all phases of digestion.

### Example 4

### Removal of the Open Pyloric Band

As in example 2, the open or helical band can be removed in a minimally invasive procedure where the endoscopically delivered forceps penetrate the submucosa and grab the end of the open band and rotate the ring out of the implanted position. The single incision in the submucosa is left to heal, sutured, stapled, welded or clipped closed.

### Example 5

### Submucosal Implantation of a Dynamic Pyloric Band

As in example 3, a space is formed between the submucosa and muscle layer of the pylorus region with a tissue separating tool. In this example, a rigid outer band with an inflatable inner lumen is then threaded around the inner circumference of the muscle layer and optionally attached to the muscle layer using sutures. The two ends of the open band are connected to form a closed band. The submucosal hole is sutured closed.

When the inner lumen is not inflated, the outer band pushes on the pyloric muscle to fix the pyloric open. When the inner lumen is inflated with saline from a reservoir connected to the device, the submucosal and mucosal folds are pushed together to close the pyloric opening. Power for the device is supplied from an electromagnetic source outside the body. After sensing a pattern of pylorus electrical activity associated with eating via pyloric electrodes on the surface of the pyloric band, the pylorus is kept open to initiate the first satiety feedback loop of excessive chyme present in the duodenum with a decreased gastric emptying lag phase. After a brief delay, the lumen is inflated and the pyloric opening is closed to initiate the second satiety feedback loop of stomach distension. Once no further ingestion is detected, the inflation of the inner lumen is then cycled to allow the ingested contents into the duodenum at a controlled rate, whereby the device acts as a prosthetic pylorus sphincter. Alternatively the device becomes passive at this stage and the normal pyloric function takes over to process the gastric contents normally. The sequence and duration of events is programmed into the device and adjusted occasionally based on patient feedback, doctor instructions, or objective feedback relating to the progress of the patient towards overcoming their GI problems or eating disorder.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A gastrointestinal device comprising a band being sized and configured for residing in or around a pyloric region, said band being for maintaining pyloric opening at a predetermined size.

2. The gastrointestinal device of claim 1, wherein said band is sized and configured for accelerating stomach emptying following ingestion of food.

3. The gastrointestinal device of claim 1, wherein said band is adapted for implantation between a submucosal layer and a muscle layer of said pyloric sphincter region.

4. The gastrointestinal device of claim 1, wherein said band is an open band.

5. The gastrointestinal device of claim 1, wherein said band does not extend into the antrum of the stomach and the duodenum when the device is implanted.

6. The gastrointestinal device of claim 4, wherein said open band includes at least one end capable of piercing tissue.

7. The gastrointestinal device of claim 1, wherein said band is composed of at least one material selected from the group consisting of a ceramic material, a polymer, and an alloy.

8. The gastrointestinal device of claim 1, wherein an internal diameter of said band is selected from a range of 10-25 mm.

9. The gastrointestinal device of claim 1, wherein said band is configured such that a diameter thereof is adjustable following implantation.

10. The gastrointestinal device of claim 1, wherein the device further comprises at least one tissue anchoring element attached to said band.

11. The gastrointestinal device of claim 1, wherein the device further comprises a valve being disposed within said band, said valve being for preventing flow from the duodenum to the stomach.

12. The gastrointestinal device of claim 1, wherein the device further comprises electrodes being disposed on, or attached to said band.

13. The gastrointestinal device of claim 1, wherein said band is composed of a plurality of wire helices.

14. The gastrointestinal device of claim 1, wherein a length of said band is selected from a range of 1-5 cm.

15. The gastrointestinal device of claim 1, wherein said band is a perforated band.

16. The gastrointestinal device of claim 3, wherein said band is adapted for implantation between a submucosal layer and a muscle layer of a region flanking said pyloric sphincter.

17. The gastrointestinal device of claim 16, further comprising structures attached to or integrated with an outer surface of said band, said structures being sized and configured for projecting into submucosal folds of said pyloric sphincter region.

18. The gastrointestinal device of claim 1, wherein said band is composed of a plurality of interlocking elements.

19. The gastrointestinal device of claim 1, wherein said band includes a fluid inflatable reservoir.

20. A method of altering eating behavior of a subject comprising fixing an opening size of a pyloric canal of the subject thereby altering the eating behavior of the subject.

21. The method of claim 20, wherein said fixing is effected by a band being sized and configured for implantation in or around a pyloric region.

22. The method of claim 20, wherein said fixing is effected by implanting a device between a submucosal layer and a muscle layer of said pyloric region.

23. The method of claim 20, wherein said fixing said opening accelerates stomach emptying.

24. The method of claim 20, wherein said fixing said opening size of said pyloric canal of the subject is effected endoscopically.

25. The method of claim 21, wherein said band includes a fluid inflatable reservoir.

26. The method of claim 21, wherein an inner diameter of said band is selected from a range of 10-25 mm.

27. The method of claim 20, wherein said opening size is adjustable following said fixing.

28. The method of claim 21, wherein a length of said band is selected from a range of 1-5 cm.

29. A device comprising an element designed and configured for placement in or around a pyloric region, the device being for shortening stomach emptying lag phase.

30. The device of claim 29, wherein the device is also capable of delaying stomach emptying.
